(19) United States
(12) Patent Application Publication
(10) Pub. No.: US 2025/0120597 A1
(43) Pub. Date: Apr. 17, 2025

(54) SPHYGMOMANOMETER AND METHOD FOR MEASURING BLOOD PRESSURE

(71) Applicant: OMRON HEALTHCARE Co., Ltd., Kyoto (JP)

(72) Inventor: Yukiya SAWANOI, Kyoto (JP)

(21) Appl. No.: 18/990,676

(22) Filed: Dec. 20, 2024

Related U.S. Application Data
(63) Continuation of application No. PCT/JP2023/018719, filed on May 19, 2023.

(30) Foreign Application Priority Data
Sep. 16, 2022 (JP) 2022-148240

(51) Int. Cl. A61B 5/021; A61B 5/022; A61B 5/025

(52) U.S. Cl. CPC A61B 5/02116; A61B 5/02233; A61B 5/025

(57) ABSTRACT

A sphygmomanometer configured to measure a blood pressure by compressing a part of a user to be measured with a cuff includes a blood pressure measurement unit configured to measure the user's blood pressure based on a pulse wave signal in a pressurization process of pressurizing a cuff pressure indicating an inner pressure of the cuff, a determination unit configured to determine whether an arrhythmia occurs in the user based on the pulse wave signal during the pressurization process, and a mode setting unit configured to set one of a first mode for executing the determining of the arrhythmia and a second mode for not executing the determining of the arrhythmia. The blood pressure measurement unit sets a first pressurization speed of the cuff pressure when the first mode is set to be lower than a second pressurization speed of the cuff pressure when the second mode is set.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/018719, filed May 19, 2023, which application claims priority to Japanese Patent Application No. 2022-148240, filed Sep. 16, 2022, which applications are incorporated herein by reference in their entireties.

### TECHNICAL FIELD

The present disclosure relates to a sphygmomanometer and a method for measuring a blood pressure.

### BACKGROUND ART

A technique for detecting atrial fibrillation using a signal acquired in the process of measuring blood pressure is known from the prior art. For example, a sphygmomanometer according to Patent Document 1 (JP 2020-192322 A) determines atrial fibrillation through data of an interval time between pulse signals of pressurization stage measurement data and data of an interval time between pulse signals of depressurization stage measurement data.

### CITATION LIST

### Patent Literature

Patent Document 1: JP 2020-192322 A

### SUMMARY OF INVENTION

### Technical Problem

As a method of determining arrhythmia using a sphygmomanometer based on the oscillometric method, a method of determining arrhythmia such as atrial fibrillation based on a pattern of pulse wave intervals acquired during a pressurization process of a cuff pressure is known. For example, a rising point or a maximum point of a pulse wave signal for each beat is detected as a feature point, and an interval between a current beat and a beat one beat before is calculated as a pulse wave interval. In order to accurately calculate the pulse wave interval, the amplitude of the acquired pulse wave signal is preferably great. Therefore, in order to increase the accuracy of the arrhythmia determination based on the pattern of the pulse wave intervals, it is necessary to increase the amplitude of the pulse wave signal to acquire a more accurate pulse wave interval.

In one aspect, an object of the present disclosure is to provide a sphygmomanometer and a method for measuring a blood pressure capable of accurately determining arrhythmia by acquiring a pulse wave signal having a large amplitude in a pressurization process of the blood pressure measurement.

### Solution to Problem

An example of the present disclosure provides a sphygmomanometer configured to measure a blood pressure by compressing a part of a user to be measured with a cuff. A sphygmomanometer includes a blood pressure measurement unit configured to measure a blood pressure of a user based on a pulse wave signal in a pressurization process of pressurizing a cuff pressure indicating an inner pressure of a cuff, a determination unit configured to determine whether or not an arrhythmia occurs in the user based on the pulse wave signal during the pressurization process, and a mode setting unit configured to set one of a first mode for executing the determining of the arrhythmia and a second mode for not executing the determining of the arrhythmia. The blood pressure measurement unit sets a first pressurization speed of the cuff pressure, when the first mode is set, to be lower than a second pressurization speed of the cuff pressure when the second mode is set.

According to the above configuration, the arrhythmia can be accurately determined by acquiring the pulse wave signal having a large amplitude in the pressurization process of the blood pressure measurement.

In another example of the present disclosure, the sphygmomanometer further includes a storage unit configured to store a pulse wave signal in a pressurization process when the blood pressure of the user is measured by the blood pressure measurement unit. The blood pressure measurement unit sets the first pressurization speed based on a pulse wave amplitude indicating an amplitude of the pulse wave signal obtained in the past and stored in the storage unit.

According to the above configuration, a more appropriate pressurization speed can be set for the user.

In another example of the present disclosure, the blood pressure measurement unit sets the first pressurization speed to be lower the smaller the pulse wave amplitude.

According to the above configuration, a highly accurate arrhythmia determination can be realized in an appropriate measurement time.

In another example of the present disclosure, the blood pressure measurement unit sets the first pressurization speed to be lower the longer a time until the cuff pressure reaches a predetermined pressure in the pressurization process.

According to the above configuration, a highly accurate arrhythmia determination can be realized in an appropriate measurement time.

In another example of the present disclosure, the blood pressure measurement unit estimates a size of the cuff based on a time until the cuff pressure reaches a predetermined pressure in the pressurization process, and sets the first pressurization speed to be lower the larger the estimated size of the cuff.

According to the above configuration, a highly accurate arrhythmia determination can be realized in an appropriate measurement time.

In another example of the present disclosure, the sphygmomanometer further includes a winding strength detection unit configured to detect a winding strength of the cuff with respect to the part of the user to be measured based on the cuff pressure in the pressurization process and a capacity change of the cuff. The blood pressure measurement unit sets the first pressurization speed to be lower the smaller the winding strength.

According to the above configuration, a highly accurate arrhythmia determination can be realized in an appropriate measurement time.

According to another example of the present disclosure, there is provided a method of measuring a blood pressure by a sphygmomanometer configured to measure a blood pressure by compressing a part of a user to be measured with a cuff. A method for measuring a blood pressure includes: measuring the blood pressure of the user based on a pulse wave signal during a pressurization process of pressurizing a cuff pressure indicating an inner pressure of the cuff; determining whether or not an arrhythmia occurs in the user based on the pulse wave signal in the pressurization process; and setting one of a first mode for executing the determining of the arrhythmia and a second mode for not executing the determining of the arrhythmia. A first pressurization speed of the cuff pressure when the first mode is set is lower than a second pressurization speed of the cuff pressure when the second mode is set.

According to the above configuration, the arrhythmia can be accurately determined by acquiring the pulse wave signal having a large amplitude in the pressurization process of the blood pressure measurement.

### Advantageous Effects of Invention

According to the present disclosure, an arrhythmia can be accurately determined by acquiring pulse wave signals having large amplitudes in a pressurization process of the blood pressure measurement.

### BRIEF DESCRIPTION OF DRAWINGS

FIG. 1 is a diagram illustrating a sphygmomanometer according to an embodiment.

FIG. 2 is a block diagram illustrating an example of a hardware configuration of the sphygmomanometer according to a first embodiment.

FIG. 3 is a block diagram illustrating a functional configuration of the sphygmomanometer according to the first embodiment.

FIG. 4 is a diagram illustrating a relationship between a pulse wave amplitude and a pressurization speed obtained in the past.

FIG. 5 is a flowchart illustrating an example of a processing procedure of the sphygmomanometer according to the first embodiment.

FIG. 6 is a flowchart illustrating another example of the processing procedure of the sphygmomanometer according to the first embodiment.

FIG. 7 is a diagram for explaining a relationship between an arrival time to a predetermined pressure and a pressurization speed.

FIG. 8 is a diagram for explaining a relationship among an arrival time until a predetermined pressure is reached, a pressurization speed, and a cuff size.

FIG. 9 is a flowchart illustrating an example of a processing procedure of the sphygmomanometer according to a second embodiment.

FIG. 10 is a flowchart illustrating another example of the processing procedure of the sphygmomanometer according to the second embodiment.

FIG. 11 is a block diagram illustrating a functional configuration of the sphygmomanometer according to a third embodiment.

FIG. 12 is a flowchart illustrating an example of a processing procedure of the sphygmomanometer according to the third embodiment.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### Application Example

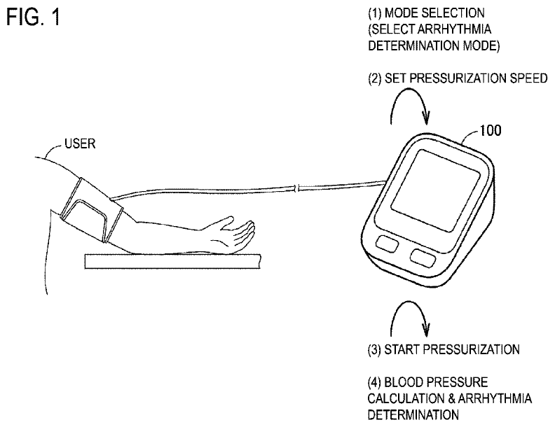

An application example of the present invention will be described with reference to FIG. 1. FIG. 1 is a diagram illustrating a sphygmomanometer 100 according to the present embodiment.

Referring to FIG. 1, the sphygmomanometer 100 is an upper-arm sphygmomanometer that measures blood pressure by compressing a part of the user to be measured (i.e., the subject) with a cuff. The sphygmomanometer 100 executes blood pressure measurement through the oscillometric method. The sphygmomanometer 100 includes a main body and a cuff (arm band) as main components. The sphygmomanometer 100 may be a wrist-type sphygmomanometer in which a main body and a cuff (arm band) are integrated. Hereinafter, the processing contents will be described with reference to FIG. 1.

In FIG. 1, a scene in which a user measures his/her blood pressure using the sphygmomanometer 100 is assumed. The sphygmomanometer 100 measures the blood pressure of the user through a pressurization measurement method of measuring the blood pressure in a pressurization process of the cuff pressure indicating the inner pressure of the cuff worn on a part of the user to be measured (e.g., an arm).

The sphygmomanometer 100 selects either a normal measuring mode (hereinafter, also simply referred to as a “normal mode”) or an arrhythmia determination mode (corresponds to (1) in FIG. 1). The arrhythmia determination mode is a mode in which the arrhythmia determination is executed together with the blood pressure measurement based on the pulse wave signal in the pressurization process of the cuff pressure. The normal mode is a mode in which only the blood pressure measurement is executed based on the pulse wave signal in the pressurization process and the arrhythmia determination is not executed. Here, it is assumed that the arrhythmia determination mode has been selected.

The sphygmomanometer 100 sets the pressurization speed of the cuff pressure in the pressurization process at the time of the arrhythmia determination mode (corresponds to (2) in FIG. 1). Here, at the time of the normal mode, the speed is set to a speed (e.g., 5.5 mmHg/s) at which a predetermined pulse rate necessary for blood pressure calculation can be secured. The pressurization speed in the arrhythmia determination mode is set to be lower than the pressurization speed in the normal mode. It is known that reducing the pressurization speed increases the amplitude of the pulse wave signal (hereinafter, “pulse wave amplitude”) obtained in the pressurization process. Therefore, the pulse wave amplitude obtained during the pressurization process at the time of the arrhythmia determination mode is larger than the pulse wave amplitude obtained during the pressurization process at the time of the normal mode.

The sphygmomanometer 100 starts pressurization of the cuff according to the set pressurization speed (corresponds to (3) in FIG. 1).

The sphygmomanometer 100 calculates the blood pressure value of the user and determines the presence or absence of occurrence of arrhythmia based on the pulse wave signal obtained in the pressurization process (corresponds to (4) in FIG. 1). In this case, the sphygmomanometer 100 displays the blood pressure value of the user and the determination result of the arrhythmia on the display.

According to the above application example, since the pulse wave amplitude obtained in the arrhythmia determination mode is larger than the pulse wave amplitude obtained in the normal mode, a more accurate pulse wave interval pattern can be obtained in the arrhythmia determination mode. Therefore, the sphygmomanometer 100 can execute a more accurate arrhythmia determination.

Note that the sphygmomanometer 100 creates a pulse wave envelope based on the amplitude of the acquired pulse wave signal and the cuff pressure, and calculates the blood pressure based on the pulse wave envelope. The sphygmomanometer 100 performs processing such as correction and smoothing of the pulse wave envelope so that the blood pressure can be calculated even when the acquired pulse wave signal is small and cannot be recognized as a pulse wave. Therefore, at the time of the normal mode in which the pressurization speed is high, the sphygmomanometer 100 can calculate the blood pressure in a short measurement time. Thus, the blood pressure measurement is quickly performed for the user who does not desire the arrhythmia determination.

As described above, in the sphygmomanometer 100 according to the present embodiment, the arrhythmia can be accurately determined by acquiring the pulse wave signal having a large amplitude during the pressurization process of the blood pressure measurement.

### Configuration Example

### First Embodiment

### (Hardware Configuration)

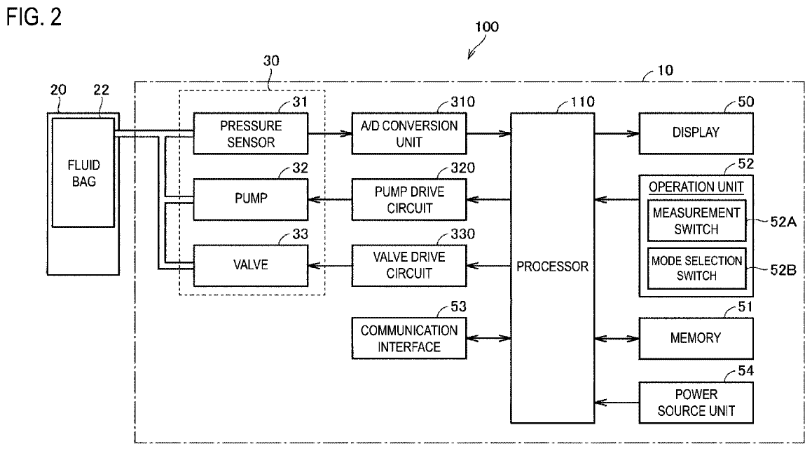

FIG. 2 is a block diagram illustrating an example of a hardware configuration of the sphygmomanometer 100 according to a first embodiment. Referring to FIG. 2, the sphygmomanometer 100 includes a main body 10 and a cuff 20 as main components. The cuff 20 interiorly includes a fluid bag 22. The main body 10 includes a processor 110, an air-system component 30 for blood pressure measurement, an A/D conversion circuit 310, a pump drive circuit 320, a valve drive circuit 330, a display 50, a memory 51, an operation unit 52, a communication interface 53, and a power source unit 54.

The processor 110 may typically be an arithmetic processing unit such as a Central Processing Unit (CPU) or a Multi Processing Unit (MPU). The processor 110 reads and executes the program stored in the memory 51 to implement each process (step) of the sphygmomanometer 100 described later. For example, the processor 110 performs control to drive the pump 32 and the valve 33 in accordance with an operation signal from the operation unit 52. In addition, the processor 110 calculates a blood pressure value using an algorithm for blood pressure calculation by an oscillometric method and displays the blood pressure value on the display 50.

The memory 51 is achieved by a Random Access Memory (RAM), a Read-Only Memory (ROM), a flash memory, or the like. The memory 51 stores a program for controlling the sphygmomanometer 100, data used for controlling the sphygmomanometer 100, setting data for setting various functions of the sphygmomanometer 100, data of a measurement result of a blood pressure value, a pulse rate, a pulse wave interval, and the like. Furthermore, the memory 51 is used as working memory and the like for when executing a program.

The air-system component 30 supplies or discharges air to or from the fluid bag 22 interiorly contained in the cuff 20 through an air line. The air-system component 30 includes a pressure sensor 31 for detecting a pressure in the fluid bag 22, and a pump 32 and a valve 33 serving as an expanding/contracting mechanism section for expanding/contracting the fluid bag 22.

The pressure sensor 31 detects a pressure (cuff pressure) in the fluid bag 22 and outputs a signal (cuff pressure signal) corresponding to the detected pressure to the A/D conversion circuit 310. The pressure sensor 31 is, for example, a piezo-resistive pressure sensor connected to the pump 32, the valve 33, and the fluid bag 22 interiorly contained in the cuff 20 via the air line. The pump 32 supplies air as a fluid to the fluid bag 22 through an air line in order to pressurize the cuff pressure. The valve 33 is opened and closed to control the cuff pressure by discharging air in the fluid bag 22 through the air line or enclosing air in the fluid bag 22.

The A/D conversion circuit 310 converts an output value of the pressure sensor 31 (e.g., a voltage value corresponding to a change in electric resistance due to a piezo-resistive effect) from an analog signal to a digital signal and outputs the converted signal to the processor 110. The processor 110 acquires a signal representing the cuff pressure according to the output value of the A/D conversion circuit 310. The pump drive circuit 320 controls driving of the pump 32 based on a control signal provided from the processor 110. The valve drive circuit 330 controls opening and closing of the valve 33 based on a control signal provided from the processor 110.

When the blood pressure is measured according to the oscillometric method, the following operation is generally performed. Specifically, a cuff is wound around a part of a subject to be measured (wrist, arm, etc.) in advance, and the cuff is pressurized by controlling the pump 32 and the valve 33 at the time of measurement. In the process of pressurization, the cuff pressure is detected by the pressure sensor, and the variation of arterial volume occurring in the artery of the part to be measured is taken out as a pulse wave signal. The systolic blood pressure and the diastolic blood pressure are calculated based on the change (mainly rise and fall) in the amplitude of the pulse wave signal accompanying the change in the cuff pressure at that time.

The operation unit 52 inputs an operation signal corresponding to an instruction by a user to the processor. The operation unit 52 includes a measurement switch 52A for receiving a blood pressure measurement instruction from the user, and a mode selection switch 52B for selecting a measuring mode.

When the measurement switch 52A is pressed, the part to be measured is temporarily compressed by the cuff 20, and the blood pressure measurement is executed through the oscillometric method. When the measurement switch 52A is pressed again during the blood pressure measurement, the blood pressure measurement is stopped.

Furthermore, when the mode selection switch 52B is pressed, the measuring mode is switched. For example, when the mode selection switch 52B is pressed in a case where the current measuring mode is set to the normal mode, the measuring mode is switched to the arrhythmia determination mode.

The display 50 displays various kinds of information including a blood pressure measurement result and the like based on a control signal from the processor 110. The communication interface 53 exchanges various kinds of information with an external device. The power source unit 54 supplies power to the processor 110 and each piece of hardware.

### (Functional Configuration)

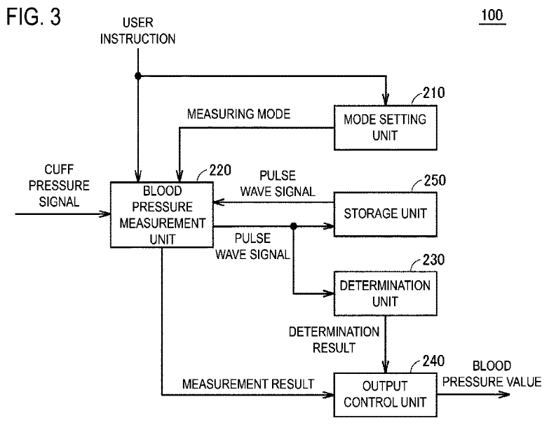

FIG. 3 is a block diagram illustrating a functional configuration of the sphygmomanometer 100 according to the first embodiment. Referring to FIG. 3, the sphygmomanometer 100 includes a mode setting unit 210, a blood pressure measurement unit 220, a determination unit 230, and an output control unit 240 as a main functional configuration. Each of these functions is realized, for example, by the processor 110 of the sphygmomanometer 100 executing a program stored in the memory 51. Note that some or all of these functions may be configured to be realized by hardware. The sphygmomanometer 100 further includes a storage unit 250. The storage unit 250 is realized by the memory 51.

The mode setting unit 210 sets one of an arrhythmia determination mode in which determination of presence or absence of arrhythmia of the user is executed and a normal mode in which determination of arrhythmia is not executed. Typically, the mode setting unit 210 sets either the arrhythmia determination mode or the normal mode in accordance with a mode selection instruction from the user via the operation unit 52 (e.g., the mode selection switch 52B).

Note that the mode setting unit 210 may be configured to automatically set either mode according to a schedule defined in advance. For example, when the blood pressure measurement is started (e.g., blood pressure measurement is started by pressing the measurement switch 52A) in the time zone H of the day, the arrhythmia determination mode is automatically set. On the other hand, when the blood pressure measurement is executed in a time zone other than the time zone H in the day, the normal mode is automatically set.

The blood pressure measurement unit 220 controls the cuff pressure in accordance with a measurement start instruction from the user via the operation unit 52 (e.g., the measurement switch 52A). Specifically, the blood pressure measurement unit 220 drives the pump 32 via the pump drive circuit 320 and performs a control for driving the valve 33 via the valve drive circuit 330. The valve 33 is opened and closed to discharge or enclose the air in the fluid bag 22 and control the cuff pressure.

The blood pressure measurement unit 220 receives the cuff pressure signal detected by the pressure sensor 31 and takes out a pulse wave signal representing the pulse wave of the part to be measured superimposed on the cuff pressure signal. That is, the blood pressure measurement unit 220 detects a pulse wave, which is a pressure component superimposed on the cuff pressure signal in synchronization with the pulsation of the heart of the user, from the cuff pressure signal.

The blood pressure measurement unit 220 calculates blood pressure information of the user based on the cuff pressure signal and the pulse wave signal superimposed on the cuff pressure signal. Specifically, the blood pressure measurement unit 220 measures the blood pressure of the user according to the oscillometric method. In the present embodiment, a pressurization measurement method of measuring the blood pressure of the user based on the pulse wave signal in the pressurization process of pressurizing the cuff pressure is adopted. Typically, the blood pressure measurement unit 220 calculates a systolic blood pressure, a diastolic blood pressure, a pulse rate, and a pulse pressure. The storage unit 250 stores information obtained at the time of blood pressure measurement (e.g., pulse wave signal, systolic blood pressure, diastolic blood pressure, pulse rate, pulse pressure, etc.).

The blood pressure measurement unit 220 sets the pressurization speed of the cuff pressure in the pressurization process based on the measuring mode set by the mode setting unit 210. Specifically, the blood pressure measurement unit 220 makes the pressurization speed G1 of the cuff pressure when the arrhythmia determination mode is set lower than the pressurization speed G2 of the cuff pressure when the normal mode is set.

Typically, the blood pressure measurement unit 220 sets the pressurization speed G2 to a speed (e.g., 5.5 mmHg/s) at which a predetermined pulse rate (e.g., 8 beats) necessary for measuring the blood pressure of the user can be secured.

As a method of setting the pressurization speed G1 by the blood pressure measurement unit 220, several methods can be considered. In one aspect, the blood pressure measurement unit 220 sets the pressurization speed G1 to a speed (e.g., 4 mmHg/s) defined in advance. This speed (e.g., 4 mmHg/s) is a speed sufficiently lower than the pressurization speed G2 set by the above method. The blood pressure measurement unit 220 executes blood pressure measurement based on the pulse wave signal in the pressurization process of the cuff pressurized at the set pressurization speed G1.

In another aspect, the blood pressure measurement unit 220 sets the pressurization speed G1 based on the amplitude (i.e., pulse wave amplitude) of the past pulse wave signal stored in the storage unit 250.

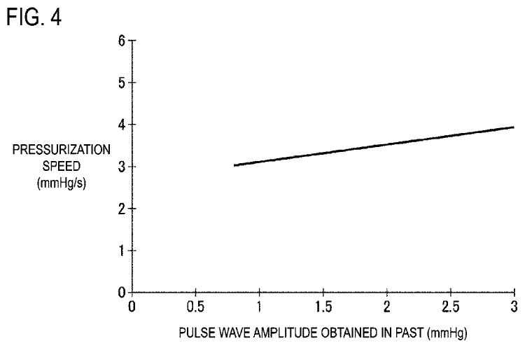

FIG. 4 is a diagram illustrating a relationship between a pulse wave amplitude and a pressurization speed obtained in the past. In the graph of FIG. 4, the vertical axis represents the pressurization speed G1, and the horizontal axis represents the pulse wave amplitude (hereinafter, also referred to as “pulse wave amplitude Am” for the sake of convenience) obtained in the past pressurization process. It can be recognized that the pressurization speed G1 is lower the smaller the pulse wave amplitude Am. This is because a highly accurate arrhythmia determination is performed by obtaining the pulse wave amplitude having a certain magnitude or more in the pressurization process.

Specifically, when the pulse wave amplitude Am obtained in the past is small, it is estimated that the pulse wave amplitude during the pressurization process at the time of the current blood pressure measurement is also small. Therefore, when the pulse wave amplitude Am obtained in the past is small, the pressurization speed is reduced so that the pulse wave amplitude obtained in the current pressurization process becomes large. On the other hand, when the pulse wave amplitude Am obtained in the past is sufficiently large, it is considered that a large pulse wave amplitude can be obtained in the current pressurization process as well, and hence the pressurization speed is increased. In this case, the measurement time is shortened. In this manner, the pressurization speed G1 is set to a pressurization speed necessary for obtaining a pulse wave amplitude of a certain magnitude or more. As a result, a more appropriate depressurization speed can be set for the user, and a highly accurate arrhythmia determination can be realized in an appropriate measurement time.

The storage unit 250 stores the pulse wave amplitude in a past predetermined period. For example, the storage unit 250 stores a pulse wave signal in the pressurization process when the blood pressure of the user is measured. As an example, the blood pressure measurement unit 220 calculates an average value of the maximum pulse wave amplitudes during the pressurization process at the time of a plurality of (e.g., three times) most recent blood pressure measurements based on the data stored in the storage unit 250, and sets the average value as the pulse wave amplitude Am. As another example, the blood pressure measurement unit 220 extracts data at the time of the past blood pressure measurement in the same time zone (e.g., morning, daytime, night, etc.) as the current blood pressure measurement, and sets the maximum pulse wave amplitude during the pressurization process at the time of the blood pressure measurement as the pulse wave amplitude Am. As still another example, the blood pressure measurement unit 220 sets, as the pulse wave amplitude Am, the maximum pulse wave amplitude during the pressurization process of a plurality of blood pressure measurements within a predetermined period (e.g., one day).

Referring to FIG. 3 again, when the arrhythmia determination mode is set, the determination unit 230 determines whether or not an arrhythmia of the user has occurred based on the pulse wave signal in the pressurization process. A known method is used for a method for determining arrhythmia. For example, the determination unit 230 determines the presence or absence of occurrence of arrhythmia based on the occurrence intervals (i.e., pulse wave interval) of the plurality of pulse waves acquired from the pulse wave signal.

The output control unit 240 displays the measurement result of the blood pressure measurement unit 220, the determination result of the determination unit 230, and the like on the display 50. Note that the output control unit 240 may transmit the measurement result and the determination result to an external device via the communication interface 53, or may be configured to output a voice via a speaker (not illustrated).

### (Processing Procedure)

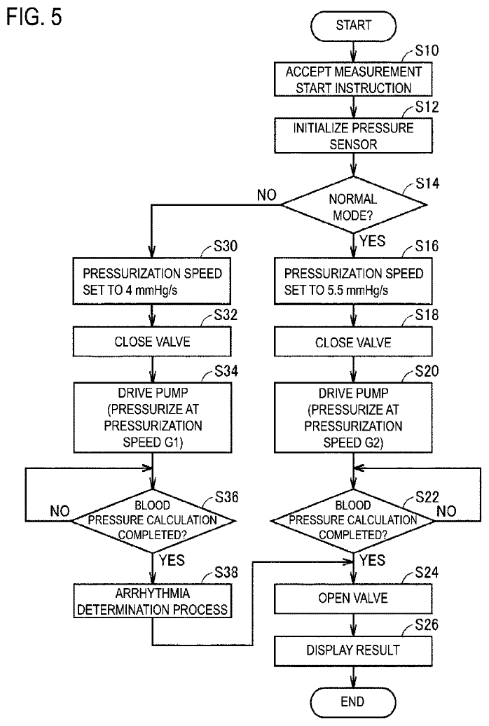

FIG. 5 is a flowchart illustrating an example of a processing procedure of the sphygmomanometer 100 according to the first embodiment. At the start of the process, it is assumed that the user is in a state of wearing the cuff 20 of the sphygmomanometer 100. In FIG. 5, it is assumed that the normal mode or the arrhythmia determination mode is set as the measuring mode.

Referring to FIG. 5, the processor 110 of the sphygmomanometer 100 accepts an instruction to start blood pressure measurement from the user via the measurement switch 52A of the operation unit 52 (step S10). The processor 110 initializes the pressure sensor 31 (step S12). Specifically, the processor 110 initializes the processing memory area, and performs 0 mmHg adjustment (setting the atmospheric pressure to 0 mmHg) of the pressure sensor 31 in a state where the pump 32 is turned off (stopped) and the valve 33 is opened. The processor 110 determines whether the currently set measuring mode is the normal mode (step S14).

First, a case where the measuring mode is the normal mode (YES in step S14) will be described. The processor 110 sets the pressurization speed G2 to a speed Ka (e.g., 5.5 mmHg/s) (step S16). The processor 110 closes the valves 33 via the valve drive circuit 330 (step S18). Subsequently, the processor 110 turns on (activates) the pump 32 via the pump drive circuit 320 and starts pressurization of the cuff 20 (fluid bag 22) at the pressurization speed G2 (step S20).

The processor 110 extracts a pulse wave signal from the cuff pressure signal detected by the pressure sensor 31, attempts to calculate the maximal blood pressure (systolic blood pressure) and the minimal blood pressure (diastolic blood pressure) based on the pulse wave signal, and determines whether or not the blood pressure calculation is completed (step S22). When the blood pressure calculation cannot be completed yet due to lack of data (NO in step S22), the processor 110 returns to step S22. When the blood pressure calculation is completed (YES in step S22), the processor 110 performs a control to stop the pump 32 and fully open the valve 33 (step S24), and rapidly exhaust the air in the cuff 20. The processor 110 displays the blood pressure value (measurement result) measured in step S22 on the display 50 (step S26).

Next, a case where the measuring mode is the arrhythmia determination mode (NO in step S14) will be described. The processor 110 sets the pressurization speed G1 to a speed Kb (e.g., 4 mmHg/s) (step S30). It is assumed that the speed Kb is a speed lower than the speed Ka (i.e., the pressurization speed G2). The processor 110 closes the valves 33 via the valve drive circuit 330 (step S32). Subsequently, the processor 110 turns on the pump 32 via the pump drive circuit 320 and starts pressurization of the cuff 20 at the pressurization speed G1 (step S34).

The processor 110 attempts to calculate the systolic blood pressure and the diastolic blood pressure based on the pulse wave signal, and determines whether or not the blood pressure calculation is completed (step S36). When the blood pressure calculation cannot be completed (NO in step S36), the processor 110 returns to step S36. When the blood pressure calculation is completed (YES in step S36), the processor 110 executes the arrhythmia determination process (step S38). Specifically, the processor 110 determines the presence or absence of occurrence of arrhythmia of the user based on the pulse wave interval acquired from the pulse wave signals in the pressurization process.

Subsequently, the processor 110 executes steps S24 and S26 described above. Note that in step S26, the blood pressure measurement result and the arrhythmia determination result are displayed as the measurement results.

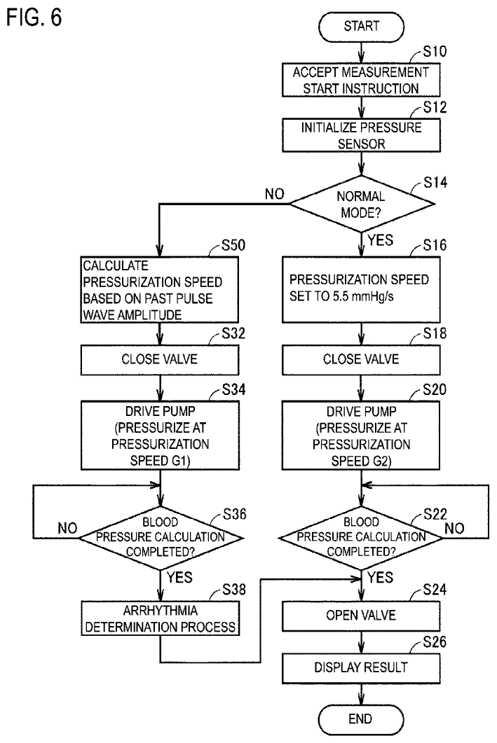

FIG. 6 is a flowchart illustrating another example of the processing procedure of the sphygmomanometer 100 according to the first embodiment. The flowchart of FIG. 6 corresponds to a flowchart in which step S30 in the flowchart of FIG. 5 is replaced with step S50. Therefore, the detailed description of the processes other than step S50 will not be repeated.

Referring to FIG. 6, in step S50, the processor 110 calculates the pressurization speed G1 based on the pulse wave amplitude obtained in the past. For example, the processor 110 calculates the pressurization speed G1 according to the graph of FIG. 4.

### Second Embodiment

In a second embodiment, a method of setting the pressurization speed G1 focusing on the size of the cuff (e.g., the volume of the fluid bag) will be described.

In order to accurately perform the blood pressure measurement, it is necessary to appropriately pressure-close the artery of the part to be measured by the cuff pressure. Therefore, the size of the cuff is determined according to the peripheral length of the part to be measured, and the size of the cuff becomes larger the longer the peripheral length of the part to be measured. Since the volume of the fluid bag increases as the size of the cuff increases, the ratio of the change in the volume of the fluid bag with respect to the change in the volume of the artery decreases. As a result, the change in the cuff pressure, i.e., the amplitude of the pulse wave signal (i.e., the pulse wave amplitude) becomes smaller. Hereinafter, a method of setting the pressurization speed G1 according to the second embodiment will be described.

### (Functional Configuration)

A functional configuration of the sphygmomanometer 100 according to the second embodiment will be described with reference to a block diagram of FIG. 3. The blood pressure measurement unit 220 according to the second embodiment further has the following functions in addition to the functions described with reference to FIG. 3.

In order to set the pressurization speed G1, the blood pressure measurement unit 220 first drives the pump 32 so as to have a constant discharge flow rate per unit time to pressurize the cuff. The blood pressure measurement unit 220 measures an arrival time T until the cuff pressure reaches a predetermined pressure in the pressurization process based on the cuff pressure signal detected by the pressure sensor 31. The arrival time T changes according to the volume (size) of the cuff. For example, the blood pressure measurement unit 220 measures the arrival time T until the cuff pressure reaches “30 mmHg” from “0 mmHg”. The blood pressure measurement unit 220 sets the pressurization speed G1 to be lower the longer the arrival time T. Then, the blood pressure measurement unit 220 executes the blood pressure measurement based on the pulse wave signal obtained during the pressurization process of pressurizing the cuff at the set pressurization speed G1.

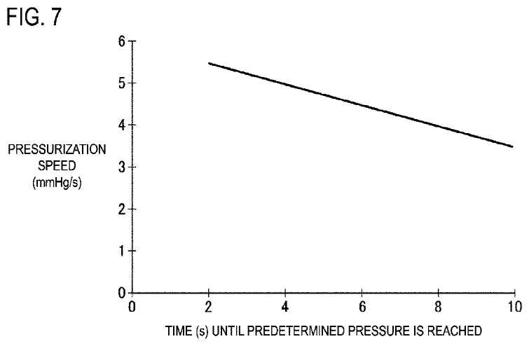

FIG. 7 is a diagram for explaining a relationship between an arrival time to a predetermined pressure and a pressurization speed. In the graph of FIG. 7, the vertical axis represents the pressurization speed G1, and the horizontal axis represents the arrival time T until the cuff pressure reaches “30 mmHg” from “0 mmHg”.

As illustrated in the graph of FIG. 7, it can be recognized that the pressurization speed G1 becomes lower the longer the arrival time T. This is because a highly accurate arrhythmia determination is performed by obtaining the pulse wave amplitude having a certain magnitude or more in the pressurization process.

Specifically, in order to set the pressurization speed G1, the blood pressure measurement unit 220 first drives the pump 32 so as to have a constant discharge flow rate per unit time to pressurize the cuff pressure. Here, the longer the arrival time T, the larger the volume of the cuff (i.e., the size of the cuff). As described above, the pulse wave amplitude becomes smaller the larger the size of the cuff. Therefore, by setting the pressurization speed G1 to be lower the longer the arrival time T (i.e., the larger the size of the cuff), the pulse wave amplitudes obtained during the pressurization process can be increased. As a result, a highly accurate arrhythmia determination is realized.

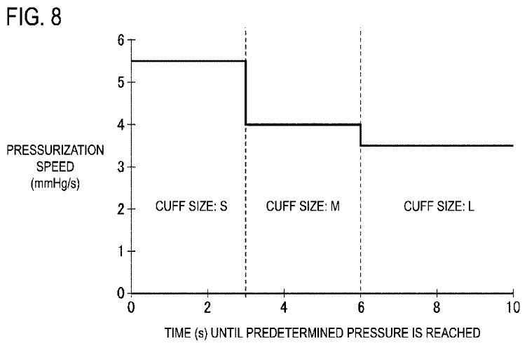

Note that the blood pressure measurement unit 220 may be configured to estimate the size of the cuff based on the arrival time T. FIG. 8 is a diagram for explaining a relationship among an arrival time until a predetermined pressure is reached, a pressurization speed, and a cuff size. In the graph of FIG. 8, the vertical axis represents the pressurization speed G1, and the horizontal axis represents the arrival time T.

The blood pressure measurement unit 220 estimates the cuff size to be “Small” when the arrival time T is less than three seconds, estimates the cuff size to be “Medium” when the arrival time T is longer than or equal to three seconds and less than six seconds, and estimates the cuff size to be “Large” when the arrival time Tis longer than or equal to six seconds. That is, the blood pressure measurement unit 220 estimates that the cuff size is larger the longer the arrival time T.

The blood pressure measurement unit 220 sets the pressurization speed G1 to 5.5 mmHg/s when the cuff size is estimated as “Small”, sets the pressurization speed G1 to 4 mmHg/s when the cuff size is estimated as “Medium”, and sets the pressurization speed G1 to 3.5 mmHg/s when the cuff size is estimated as “Large”. That is, the blood pressure measurement unit 220 sets the pressurization speed G1 to be lower the larger the cuff size.

### (Processing Procedure)

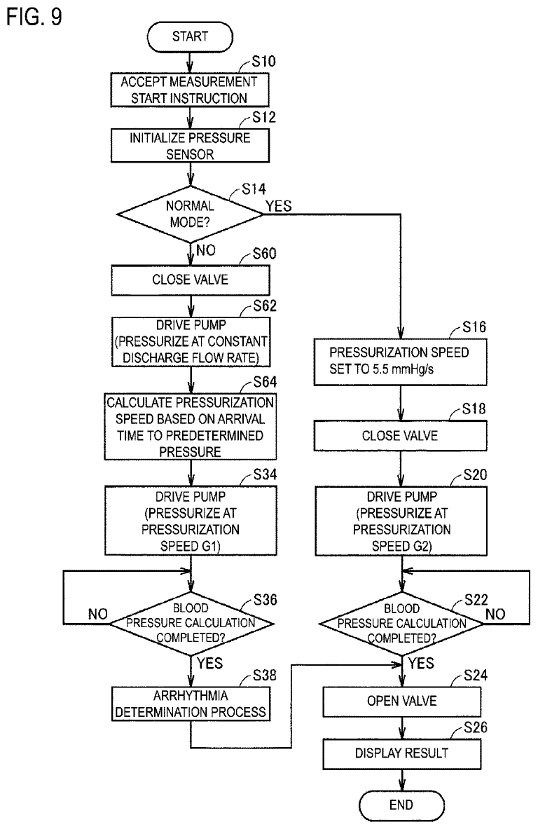

FIG. 9 is a flowchart illustrating an example of a processing procedure of a sphygmomanometer 100 according to a second embodiment. The flowchart of FIG. 9 is different from the flowchart of FIG. 5 in the processes of steps S60 to S64 after the arrhythmia determination mode is selected in step S14 (when NO in step S14). Therefore, the detailed description of the processes other than steps S60 to S64 will not be repeated.

The processor 110 closes the valve 33 via the valve drive circuit 330 (step S60), turns on the pump 32 via the pump drive circuit 320, and pressurizes the cuff at a constant discharge flow rate (step S62). The processor 110 measures the arrival time T until the cuff pressure reaches the predetermined pressure, and calculates the pressurization speed G1 based on the arrival time T (step S64). For example, the processor 110 calculates the pressurization speed G1 according to the graph of FIG. 7. The process after step S34 is similar to that in FIG. 5.

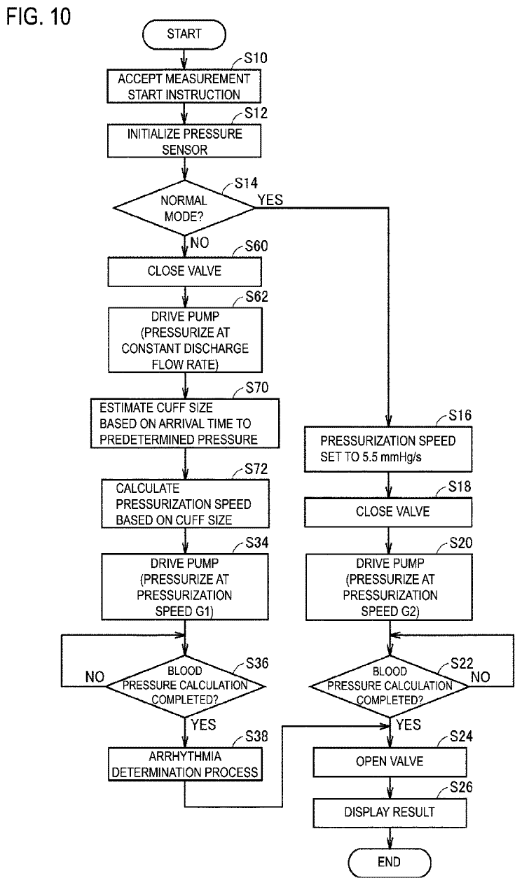

FIG. 10 is a flowchart illustrating another example of the processing procedure of the sphygmomanometer 100 according to the second embodiment. The flowchart of FIG. 10 corresponds to a flowchart in which step S64 in the flowchart of FIG. 9 is replaced with steps S70 and S72. Therefore, the detailed description of the processes other than steps S70 and S72 will not be repeated.

Referring to FIG. 10, the processor 110 estimates the cuff size based on the arrival time T (step S70), and calculates the pressurization speed G1 based on the estimated cuff size (step S72). Specifically, the processor 110 estimates the cuff size according to the graph of FIG. 8 and calculates the pressurization speed G1.

### Third Embodiment

In a third embodiment, a method of setting the pressurization speed G1 focusing on the tightening degree of the cuff will be described.

When the cuff is loosely wound, the fluid bag needs to be excessively inflated until the gap between the living body and the cuff is filled. Therefore, in a case where the cuff is loosely wound, the volume of the fluid bag becomes larger than that in a case where the cuff is tightly wound, and as a result, the amplitude of the obtained pulse wave signal becomes small. Hereinafter, a method of setting the pressurization speed G1 according to the third embodiment will be described.

### (Functional Configuration)

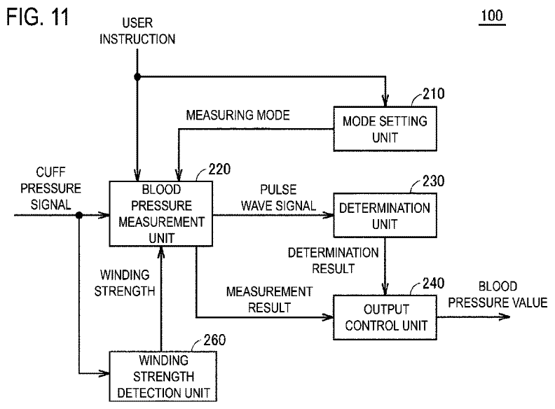

FIG. 11 is a block diagram illustrating a functional configuration of the sphygmomanometer 100 according to the third embodiment. Referring to FIG. 11, the sphygmomanometer 100 includes a mode setting unit 210, a blood pressure measurement unit 220, a determination unit 230, an output control unit 240, and a winding strength detection unit 260. Each of these functions is realized, for example, by the processor 110 of the sphygmomanometer 100 executing a program stored in the memory 51. The functions of the mode setting unit 210, the determination unit 230, and the output control unit 240 are similar to the functions described with reference to FIG. 3.

The winding strength detection unit 260 detects the winding strength of the cuff 20 with respect to the part to be measured based on the cuff pressure in the pressurization process and the change in the capacity of the cuff 20. The winding strength detection method is realized by a known method. Typically, the winding strength detection unit 260 has a similar function as the winding strength detection unit described in JP 5408142.

As an example, in the pressurization process of pressurizing the cuff by driving the pump 32 so as to have a constant discharge flow rate, the winding strength detection unit 260 compares the value of the capacity change of the cuff 20 detected with the change in the cuff pressure from the pressure P1 to the pressure P2 with the value of the capacity change of the cuff 20 detected with the change in the cuff pressure from the pressure P2 to the pressure P2 based on the pressure-capacity change relationship indicated by the capacity change of the cuff 20 detected with the change in the cuff pressure from the pressure P1 to the pressure P3 and the capacity change of the cuff 20 detected with the change in the cuff pressure from the pressure P2 to the pressure P3, and detects the winding strength of the cuff 20 from the comparison result.

The pressures P1, P2, and P3 are cuff pressures suitable for detecting the winding strength of the cuff determined in advance by an experiment or the like. Time when the cuff pressure reaches the pressures P1 to P3 is defined as time V1 to V3, respectively.

In this case, the winding strength detection unit 260 calculates a change ΔP12, which is a difference from the pressure P1 to the pressure P2, and a time ΔV12 indicating a time required for the cuff pressure to change by ΔP12 (i.e., the time V2-V1). The time ΔV12 is proportional to the change in fluid volume in the cuff as the pressure changes from the pressure P1 to the pressure P2. In this case, the winding strength detection unit 260 calculates a change ΔP23, which is a difference from the pressure P2 to the pressure P3, and a time ΔV23 indicating a time required for the cuff pressure to change by ΔP23 (i.e., the time V3-V2). The time ΔV23 is proportional to the change in fluid volume in the cuff as the pressure changes from the pressure P2 to the pressure P3.

The winding strength detection unit 260 calculates the pressure capacity change indexes ΔP12/ΔV12 and ΔP23/ΔV23, and compares the calculated values of both. The winding strength detection unit 260 detects the winding strength based on the comparison result.

In a case where the comparison result is (ΔP12/ΔV12)< (ΔP23/ΔV23), the winding strength detection unit 260 detects that the winding strength of the cuff 20 is “loose winding”. In a case where the comparison result is (ΔP12/ΔV12)> (ΔP23/ΔV23), the winding strength detection unit 260 detects that the winding strength of the cuff 20 is “tight winding”. In a case where the comparison result is (ΔP12/ΔV12)=(ΔP23/ΔV23), the winding strength detection unit 260 detects that the winding strength of the cuff 20 is “exact winding”.

“Loose winding” is a winding state in which the cuff 20 is loosely wound around the part to be measured and the pressurization on the part to be measured is lower than an appropriate level. “Exact winding” is a winding state in which the cuff 20 is appropriately wound around the part to be measured and the pressurization on the part to be measured is an appropriate level. “Tight winding” is a winding state in which the cuff 20 is loosely wound around the part to be measured and the pressurization on the part to be measured is higher than an appropriate level.

The blood pressure measurement unit 220 sets the pressurization speed G1 to be lower the smaller the winding strength detected by the winding strength detection unit 260. For example, for the sake of convenience, the pressurization speed G1 in the case where the winding strength is “loose winding” is set to “G1a”, the pressurization speed G1 in the case where the winding strength is “exact winding” is set to “G1b”, and the pressurization speed G1 in the case where the winding strength is “tight winding” is set to “G1c”. In this case, the pressurization speed G1a is the lowest, the pressurization speed G1c is the fastest, and the pressurization speed G1b is intermediate (i.e., G1a<G1b<G1c).

### (Processing Procedure)

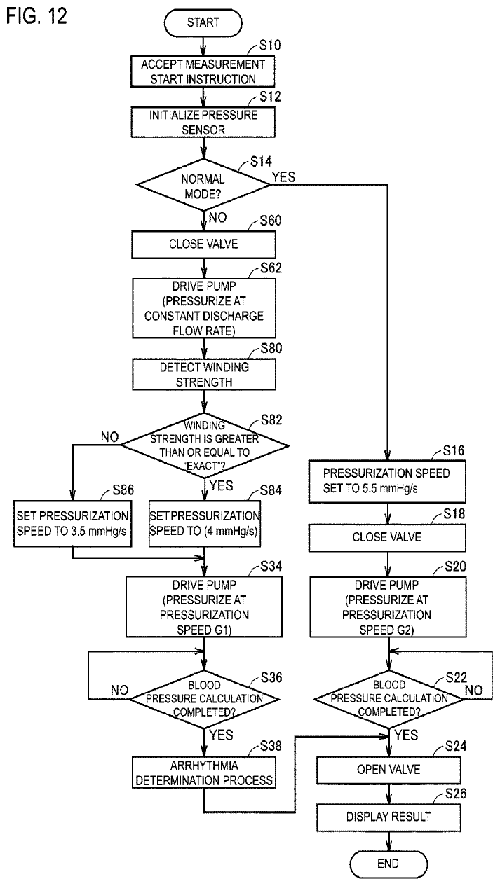

FIG. 12 is a flowchart illustrating an example of a processing procedure of the sphygmomanometer 100 according to the third embodiment. The flowchart of FIG. 12 corresponds to a flowchart in which step S64 in the flowchart of FIG. 9 is replaced with steps S80, S82, S84, and S86. Therefore, the detailed description of the processes other than steps S80, S82, S84, and S86 will not be repeated.

Referring to FIG. 12, the processor 110 detects the winding strength of the cuff 20 with respect to the part to be measured (step S80). The processor 110 determines whether or not the detected winding strength is greater than or equal to “exact winding” (i.e., “exact winding” or “tight winding”) (step S82). When the winding strength is greater than or equal to “exact winding” (YES in step S82), the processor 110 sets the pressurization speed G1 to the speed Kb (e.g., 4 mmHg/s) (step S84). The speed Kb is a speed lower than the speed Ka set as the pressurization speed G2 in step S16. When the winding strength is “loose winding” (NO in step S82), the processor 110 sets the pressurization speed G1 to the speed Kc (e.g., 3.5 mmHg/s) (step S86). The speed Kc is a speed lower than the speed Kb.

In the flowchart of FIG. 12, the configuration in which the pressurization speed G1 is set as the speed Kb in a case where the winding strength is greater than or equal to “exact winding” has been described, but this configuration is not the sole case. For example, a configuration may be adopted in which the pressurization speed G1 is set to the pressurization speeds G1a to G1c described above in accordance with the winding strength (“loose winding”, “exact winding”, or “tight winding”).

### Other Embodiments

(1) In the above-described embodiment, a program may be provided that causes a computer to function and execute controls such as those described in the flowcharts described above. Such a program can also be provided as a program product recorded on a non-temporary computer-readable recording medium attached to a computer, such as a flexible disk, a compact disc read only memory (CD-ROM), a secondary storage device, a main storage device, and a memory card. Alternatively, a program may be provided by recording the program on a recording medium such as a hard disk built into a computer. The program may also be provided by download via a network.

(2) The configuration exemplified as the embodiment described above is an example of a configuration of the present invention, and the configuration can be combined with other known technology, and one part thereof may be omitted or modified within the scope not deviating from the gist of the present invention. Furthermore, the processes and configurations described in other embodiments may be employed as appropriate in the embodiments described above.

### Supplementary Notes

As described above, the present embodiments include the following disclosures.

### [Configuration 1]

A sphygmomanometer (100) configured to measure a blood pressure by compressing a part of a user to be measured with a cuff (20), the sphygmomanometer including: a blood pressure measurement unit (220) configured to measure the blood pressure of the user based on a pulse wave signal in a pressurization process of pressurizing a cuff pressure indicating an inner pressure of the cuff; a determination unit (230) configured to determine whether or not an arrhythmia occurs in the user based on the pulse wave signal during the pressurization process; and a mode setting unit (210) configured to set one of a first mode for executing the determining of the arrhythmia and a second mode for not executing the determining of the arrhythmia, wherein the blood pressure measurement unit sets a first pressurization speed of the cuff pressure when the first mode is set to be lower than a second pressurization speed of the cuff pressure when the second mode is set.

### [Configuration 2]

The sphygmomanometer according to configuration 1, further including: a storage unit (250) configured to store a pulse wave signal in the pressurization process when the blood pressure of the user is measured by the blood pressure measurement unit, wherein the blood pressure measurement unit sets the first pressurization speed based on a pulse wave amplitude indicating an amplitude of the pulse wave signal obtained in the past and stored in the storage unit.

### [Configuration 3]

The sphygmomanometer according to the configuration 2, wherein the blood pressure measurement unit sets the first pressurization speed to be lower the smaller the pulse wave amplitude.

### [Configuration 4]

The sphygmomanometer according to configuration 1, wherein the blood pressure measurement unit sets the first pressurization speed to be lower the longer a time until the cuff pressure reaches a predetermined pressure in the pressurization process.

### [Configuration 5]

The sphygmomanometer according to the configuration 1, wherein the blood pressure measurement unit estimates a size of the cuff based on a time until the cuff pressure reaches a predetermined pressure in the pressurization process, and sets the first pressurization speed to be lower the larger the estimated size of the cuff.

### [Configuration 6]

The sphygmomanometer according to the configuration 1, further including: a winding strength detection unit (260) configured to detect a winding strength of the cuff with respect to the part of the user to be measured based on the cuff pressure and a capacity change of the cuff in the pressurization process, wherein the blood pressure measurement unit sets the first pressurization speed to be lower the smaller the winding strength.

### [Configuration 7]

A method for measuring a blood pressure by a sphygmomanometer (100) configured to measure a blood pressure by compressing a part of a user to be measured with a cuff (20), the method including: measuring the blood pressure of the user based on a pulse wave signal in a pressurization process of pressurizing a cuff pressure indicating an inner pressure of the cuff; determining whether or not an arrhythmia occurs in the user based on the pulse wave signal during the pressurization process; and setting one of a first mode for executing the determining of the arrhythmia and a second mode for not executing the determining of the arrhythmia, wherein a first pressurization speed of the cuff pressure when the first mode is set is lower than a second pressurization speed of the cuff pressure when the second mode is set.

The embodiments disclosed herein are illustrative in all respects and are not intended as limitations. The scope of the present invention is indicated not by the descriptions above but by the claims and includes all meaning equivalent to the scope and changes within the scope.

### REFERENCE SIGNS LIST

10 Main body, 20 Cuff, 22 Fluid bag, 30 Air-system component, 31 Pressure sensor, 32 Pump, 33 Valve, 50 Display, 51 Memory, 52 Operation unit, 52A Measurement switch, 52B Mode selection switch, 53 Communication interface, 54 Power source unit, 100 Sphygmomanometer, 110 Processor, 210 Mode setting unit, 220 Blood pressure measurement unit, 230 Determination unit, 240 Output control unit, 250 Storage unit, 260 Winding strength detection unit, 310 A/D conversion circuit, 320 Pump drive circuit, 330 Valve drive circuit.

1. A sphygmomanometer configured to measure a blood pressure by compressing a part of a user to be measured with a cuff, the sphygmomanometer comprising:
a blood pressure measurement unit configured to measure the blood pressure of the user based on a pulse wave signal in a pressurization process of pressurizing a cuff pressure indicating an inner pressure of the cuff;
a determination unit configured to determine whether or not an arrhythmia occurs in the user based on the pulse wave signal during the pressurization process; and
a mode setting unit configured to set one of a first mode for executing the determining of the arrhythmia and a second mode for not executing the determining of the arrhythmia, wherein
the blood pressure measurement unit sets a first pressurization speed of the cuff pressure when the first mode is set to be lower than a second pressurization speed of the cuff pressure when the second mode is set.

2. The sphygmomanometer according to claim 1, further comprising:
a storage unit configured to store a pulse wave signal in the pressurization process when the blood pressure of the user is measured by the blood pressure measurement unit, wherein
the blood pressure measurement unit sets the first pressurization speed based on a pulse wave amplitude indicating an amplitude of the pulse wave signal obtained in the past and stored in the storage unit.

3. The sphygmomanometer according to the claim 2, wherein
the blood pressure measurement unit sets the first pressurization speed to be lower the smaller the pulse wave amplitude.

4. The sphygmomanometer according to claim 1, wherein
the blood pressure measurement unit sets the first pressurization speed to be lower the longer a time until the cuff pressure reaches a predetermined pressure in the pressurization process.

5. The sphygmomanometer according to the claim 1, wherein
the blood pressure measurement unit
estimates a size of the cuff based on a time until the cuff pressure reaches a predetermined pressure in the pressurization process, and
sets the first pressurization speed to be lower the larger the estimated size of the cuff.

6. The sphygmomanometer according to the claim 1, further comprising:
a winding strength detection unit configured to detect a winding strength of the cuff with respect to the part of the user to be measured based on the cuff pressure and a capacity change of the cuff in the pressurization process, wherein
the blood pressure measurement unit sets the first pressurization speed to be lower the smaller the winding strength.

7. A method for measuring a blood pressure by a sphygmomanometer configured to measure a blood pressure by compressing a part of a user to be measured with a cuff, the method comprising:
measuring the blood pressure of the user based on a pulse wave signal in a pressurization process of pressurizing a cuff pressure indicating an inner pressure of the cuff;
determining whether or not an arrhythmia occurs in the user based on the pulse wave signal in the pressurization process; and
setting one of a first mode for executing the determining of the arrhythmia and a second mode for not executing the determining of the arrhythmia, wherein
a first pressurization speed of the cuff pressure when the first mode is set is lower than a second pressurization speed of the cuff pressure when the second mode is set.
